(19) United States
(12) Patent Application Publication
(10) Pub. No.: US 2025/0345376 A1
(43) Pub. Date: Nov. 13, 2025

(54) METHOD FOR ALLEVIATING PARTICULATE MATTER-INDUCED LUNG INJURY CAUSED BY PARTICULATE MATTER WITH DIAMETER OF NOT GREATER THAN 2.5 µm (PM2.5) USING ISOLATED STRAIN OF LACTOBACILLUS ACIDOPHILUS TW01

(71) Applicant: I EATING LIGHT LTD., Nantou City (TW)

(72) Inventors: Hsin-Hua CHIANG, Kaohsiung City (TW); Bo-Jyun LIOU, Kaohsiung City (TW)

(21) Appl. No.: 18/814,089

(22) Filed: Aug. 23, 2024

(30) Foreign Application Priority Data
May 13, 2024 (TW) 113117597

(51) Int. Cl. A61K 35/747; A61P 11/00

(52) U.S. Cl. CPC A61K 35/747; A61P 11/00

(57) ABSTRACT

A method for alleviating particulate matter-induced lung injury includes administering to a subject in need thereof a composition including an isolated strain of Lactobacillus acidophilus TW01, which is deposited at the Deutsche Sammlung von Mikroorganismen und Zellkulturen (DSMZ) GmbH under an accession number DSM 33990 in accordance with the Budapest Treaty.

### CROSS-REFERENCE TO RELATED APPLICATION

This application claims priority to Taiwanese Invention Patent Application No. 113117597, filed on May 13, 2024, the entire disclosure of which is incorporated by reference herein.

### FIELD

The disclosure relates to a method for alleviating particulate matter-induced lung injury using a composition that includes an isolated strain of Lactobacillus acidophilus TW01.

### BACKGROUND

Atmospheric particulate matter (also known as particulate matter (PM) or fine particles) is a major source of air pollution, and refers to tiny solid particles suspended in the atmospheric environment. The atmospheric particulate matter can be distinguished based on a size of aerodynamic diameter thereof. For example, PM₁₀ and PM_{2.5} respectively denote the particulate matter with an aerodynamic diameter of less than or equal to 10 μm and the particulate matter with an aerodynamic diameter of less than or equal to 2.5 μm.

Sources of the particulate matter include natural products, such as sea salt or volcanic eruptions, and man-made products, such as cigarette smoke, industrial emissions, combustion emissions, or motor-vehicle emissions. In addition, components of the particulate matter may include aromatic hydrocarbons, metals, minerals, organic toxins, and so forth, which have adverse effects on a respiratory system and a circulatory system of a human body. Exposure of human lungs to the particulate matter for a long period of time can cause lung injury, which may further lead to asthma, pulmonary fibrosis, and impairment of lung immune system, and in a severe case, may even develop into lung cancer.

The therapeutic efficacy of conventional methods used clinically to treat particulate matter (PM)-induced lung injury is still not ideal. Therefore, researchers in this field are committed to developing a medication that can be used to alleviate PM-induced lung injury.

As disclosed in U.S. Pat. No. 11,690,883 B2, the applicant isolated Lactobacillus acidophilus TW01 (DSM 33990) from a fermented broth of coffee grounds, and found through experimentation that Lactobacillus acidophilus TW01 could achieve an anti-inflammatory effect by regulating inflammatory cytokines in macrophages.

In spite of the aforesaid, there is still a need to develop an effective way for alleviating PM-induced lung injury.

### SUMMARY

Accordingly, an object of the present disclosure is to provide a method for alleviating particulate matter-induced lung injury, which can alleviate at least one of the drawbacks of the prior art, and which includes administering to a subject in need thereof a composition including an isolated strain of Lactobacillus acidophilus TW01. The isolated strain of Lactobacillus acidophilus TW01 is deposited at the Deutsche Sammlung von Mikroorganismen und Zellkulturen (DSMZ) GmbH under an accession number DSM 33990 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

The FIGURE shows the cell viability percentage determined in each group as described in Section A of Example 1, infra, in which the symbol “***” represents p<0.001 (compared with the normal control group), and the symbol “##” represents p<0.01 (compared with the pathological control group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

By conducting research, the applicant found that Lactobacillus acidophilus TW01 is capable of protecting human bronchial epithelial cells against cell death induced by particulate matter (PM), and hence is expected to be effective in alleviating particulate matter (PM)-induced lung injury.

Therefore, the present disclosure provides a method for alleviating particulate matter-induced lung injury, which includes administering to a subject in need thereof a composition including an isolated strain of Lactobacillus acidophilus TW01. The isolated strain of Lactobacillus acidophilus TW01 is deposited at the Deutsche Sammlung von Mikroorganismen und Zellkulturen (DSMZ) GmbH under an accession number DSM 33990 in accordance with the Budapest Treaty.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “particulate matter-induced lung injury” refers to an injury or damage to a lung of a subject after exposure to particulate matter, which includes, but is not limited to, interstitial thickening, structure distortion, abnormal collagen deposition, and pulmonary fibrosis.

As used herein, the term “particulate matter” is intended to cover particulate matter with different aerodynamic diameters, which includes, but is not limited to, particulate matter with a diameter of not greater than 10 μm (PM₁₀), particulate matter with a diameter of not greater than 2.5 μm (PM_{2.5}), and particulate matter with a diameter of not greater than 0.1 μm (PM_{0.1}). In certain embodiments, the particulate matter-induced lung injury may be caused by the particulate matter with the diameter of not greater than 2.5 μm (PM_{2.5}).

As used herein, the terms “administering” and “administration” can be interchangeably used, and mean introducing, providing or delivering a pre-determined active ingredient (e.g., the above-mentioned composition) to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the isolated strain of Lactobacillus acidophilus TW01 may be live cells or dead cells, concentrated or non-concentrated, a liquid, a paste, a semi-solid, a solid (e.g., a pellet, a granule, or a powder), and may be heat-killed, frozen, dried, or freeze-dried (e.g., may be in freeze-dried form or spray/fluid bed dried form). In an exemplary embodiment, the isolated strain of Lactobacillus acidophilus TW01 is present in the form of live cells.

According to the present disclosure, the isolated strain of Lactobacillus acidophilus TW01 may be prepared as a bacterial suspension having a bacterial concentration ranging from 10⁴ CFU/mL to 10⁹ CFU/mL. In certain embodiments, the bacterial suspension may have a bacterial concentration ranging from 10⁴ CFU/mL to 10⁶ CFU/mL. In an exemplary embodiment, the bacterial suspension may have a bacterial concentration of 10⁴ CFU/mL. In still an exemplary embodiment, the bacterial suspension may have a bacterial concentration of 10⁶ CFU/mL.

According to the present disclosure, the composition may be formulated as a food product using a standard technique well known to one of ordinary skill in the art. For example, the composition may be directly added to an edible material or may be used to prepare an intermediate composition (e.g., a food additive) suitable to be subsequently added to the edible material.

As used herein, the term “food product” refers to any article or substance that can be ingested by a subject into the body thereof. Examples of the food product may include, but are not limited to, milk powder, fermented milk, yogurt, butter, beverages (e.g., tea, coffee, etc.), functional beverages, flour products, baked foods, confectionery, candies, fermented foods, animal feeds, health foods, and dietary supplements.

According to the present disclosure, the composition may be prepared in the form of a pharmaceutical composition.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may be formulated into a suitable dosage form for oral, parenteral, inhalational, or topical administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intrasynovial injection, intrathecal injection, intraepidermal injection, subcutaneous injection, intradermal injection, intralesional injection, and sublingual administration.

For inhalational administration, the pharmaceutical composition according to the present disclosure may be formulated into a spray, e.g., a nasal spray or an oral spay, and may be administered via one of the following inhalation routes: oral inhalation and nasal inhalation.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, and bandages.

The dose and frequency of administration of the pharmaceutical composition according to the present disclosure may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials:

1. Lactobacillus acidophilus TW01

Lactobacillus acidophilus TW01 (which is disclosed in the applicant's patent TW 1788840 B) is known and readily available to the public, and has been deposited at the Bioresource Collection and Research Center (BCRC) of the Food Industry Research and Development Institute (FIRDI) (No. 331, Shih-Pin Rd., Hsinchu City 300, Taiwan) under an accession number BCRC 911039 since Mar. 5, 2021, and has also been deposited at the Deutsche Sammlung von Mikroorganismen und Zellkulturen (DSMZ) GmbH (Inhoffenstraße 7B, 38124 Braunschweig, Germany) under an accession number DSM 33990 since Aug. 2, 2021 in accordance with the Budapest Treaty.

2. Preparation of Bacterial Suspension of Lactobacillus acidophilus TW01

First, Lactobacillus acidophilus TW01 described in Section 1 of General Experimental Materials was inoculated into a Lactobacilli MRS broth (Difco Laboratories, U.S.A.), and was then cultivated under an anaerobic condition in an incubator at 37° C. for 24 hours to obtain a culture. After centrifugation at 3000 rpm for 20 minutes, the resultant cell pellet was collected, and then washed with a suitable amount of Dulbecco's phosphate buffered saline (DPBS) (Gibco), followed by centrifugation at 3000 rpm for 20 minutes. The aforesaid washing and centrifugation steps were repeated twice. After removal of the supernatant, the bacterial cells were re-suspended with a suitable amount of Bronchial Epithelial Cell Medium (BEpiCM) (ScienCell Research Laboratories (ScienCell); Cat. No. 3211) to obtain a suspension, followed by separating the suspension into a first suspension and a second suspension, and adjusting the bacterial concentration of each of the first suspension and the second suspension, thereby obtaining a first bacterial suspension having a bacterial concentration of 10⁴ CFU/mL (abbreviated as bacterial suspension 1) and a second bacterial suspension having a bacterial concentration of 10⁶ CFU/mL (abbreviated as bacterial suspension 2).

### 3. Source and Cultivation of Cell Lines

The human bronchial epithelial cells (HBEpiC) and the human colon adenocarcinoma cell line Caco-2 used in the following examples were purchased from ScienCell, U.S.A. (Cat. No. 3210) and the Bioresource Collection and Research Center (BCRC) of the Food Industry Research and Development Institute (FIRDI) (No. 331, Shih-Pin Rd., Hsinchu City 300, Taiwan), respectively.

Each of the cell lines was cultivated in a 10-cm Petri dish (Corning) containing a corresponding medium shown in Table 1 below, and then incubated in an incubator with culture conditions set at 37° C. and 5% CO₂. Medium change was performed every two to three days. Cell passage was performed when the cultured cells reached approximately 85% to 90% of confluence.

TABLE 1

| Cell line | Medium |
|---|---|
| HBEpiC | Bronchial Epithelial Cell Medium (BEpiCM) (ScienCell; Cat. |
| | No. 3211) |
| Caco-2 | Dulbecco's Modified Eagle's Medium (DMEM) (Corning, |
| | U.S.A.) supplemented with 10% fetal bovine serum (FBS) |
| | (Corning), 1 mM pyruvate (Corning), and 0.01 mg/ml human |
| | transferrin (Sigma-Aldrich Chemical Co.) |

### 4. Preparation of Cigarette Smoke Extract (CSE)

The preparation of a CSE was carried out with reference to the method described in Cheng M. Y. et al. (2016), Exp. Ther. Med., 12:4168-4174. Briefly, a cigarette (containing 0.9 mg of nicotine and 10 mg of tar) was lit, so as to generate a cigarette smoke, and then the cigarette smoke was introduced into 20 mL of the BEpiCM (ScienCell; Cat. No. 3211) using a syringe pump until the cigarette smoke was completely dissolved in the 20 mL of the BEpiCM, thereby obtaining a mixture. Thereafter, the mixture was subjected to filtration using a filter with a pore size of 0.22 μm, followed by collecting a filtrate, thereby obtaining the CSE.

### General Procedures:

### 1. Statistical Analysis:

All the experiments described below were performed in triplicates.

The experimental data obtained in all the test groups are expressed as mean±standard deviation (SD), and were analyzed using one-way analysis of variance (ANOVA) followed by Tukey's test, so as to evaluate the differences between the groups. Statistical significance is indicated by p<0.05.

### Example 1. Evaluation of the Efficacy of Lactobacillus acidophilus TW01 in Alleviating Particulate Matter (PM)-Induced Lung Injury

In order to simulate the effect of probiotic bacteria (i.e., Lactobacillus acidophilus TW01) on the lungs via intestines, in this example, the applicant established a lung model of non-contact co-cultivation system using the HBEpiC and the Caco-2 cells, generally with reference to the method described in Nishitani Y. and Mizuno M. (2010), Biosci. Microflora, 29:169-178.

a. Effect of Lactobacillus acidophilus TW01 Against PM-Induced Cell Death

### Experimental Procedures:

First, the CSE obtained in Section 4 of the General Experimental Materials was diluted using a suitable amount of the BEpiCM, so as to obtain a CSE solution having a CSE concentration of 20 vol %.

Subsequently, Transwell inserts (Corning; R3402), each of which had a polycarbonate membrane with a pore size of 3 μm and contained 0.5 mL of the DMEM, were placed in respective wells of a 12-well culture plate, and then the Caco-2 cells obtained in Section 3 of the General Experimental Materials were divided into four groups, including a normal control group, a pathological control group, and two experimental groups (i.e., experimental groups 1 and 2). Each group of the Caco-2 cells was seeded at a concentration of 1×10⁵ cells/well in a respective one of the Transwell inserts, followed by cultivation in an incubator with culture conditions set at 37° C., 5% CO₂ for 28 days. Medium change was performed every two days.

Next, an epithelial voltohmmeter (EVOM2) (World Precision Instruments, Sarasota, FL, U.S.A.) equipped with an STX2 electrode was used to measure the level of transepithelial electrical resistance (TEER) of the Caco-2 cells in each of the Transwell inserts in accordance with the manufacturer's instructions, so as to ensure that the level of TEER measured is greater than 350 Ω/cm². After that, the HBEpiC obtained in Section 3 of the General Experimental Materials were divided into four groups, including a normal control group, a pathological control group, and two experimental groups (i.e., experimental groups 1 and 2). Each group of the HBEpiC was seeded at a concentration of 1.5×10⁵ cells/well in the respective well of the 12-well culture plate, followed by co-cultivation with the Caco-2 cells of a corresponding group in an incubator (37° C., 5% CO₂) overnight.

Afterward, the HBEpiC in each of the pathological control group, and the experimental groups 1 and 2 were treated with 1 mL of the aforesaid CSE solution, and simultaneously, the Caco-2 cells in each of the experimental group 1 and the experimental group 2 were respectively treated with 0.5 mL of the bacterial suspension 1 (having the bacterial concentration of 10⁴ CFU/mL) and 0.5 mL of the bacterial suspension 2 (having the bacterial concentration of 10⁶ CFU/mL) obtained in Section 2 of the General Experimental Materials. The Caco-2 cells in the pathological control group received no treatment. Both the HBEpiC and the Caco-2 cells in the normal control group received no treatment.

After cultivation in an incubator (37° C., 5% CO₂) for 24 hours, the Transwell inserts and the liquid in the well of each group were removed, and then a suitable amount of {3-[4,5-Dimethylthiazol-2-yl]-2,5-diphenyltetrazolium bromide}(MTT, 0.5 mg/mL) (Sigma-Aldrich Chemical Co.) was added thereto, followed by incubation at 37° C. in dark for 4 hours.

Subsequently, the liquid in each well was removed, and the respective well was washed with the DPBS, followed by adding 1 mL of dimethyl sulfoxide (DMSO) (Sigma-Aldrich Chemical Co.) to be well mixed, so as to obtain a mixture. After that, the mixture in each well was subjected to determination of absorbance at a wavelength of 560 nm (OD₅₆₀) using an ELISA reader (BioTek Epoch, U.S.A.).

The cell viability percentage (%) of each group was calculated using the following Equation (1):

$$A = ( B / C ) \times 100 \qquad ( 1 )$$

where A=cell viability percentage (%)
   B=OD₅₆₀ value determined in each group
   C=OD₅₆₀ value determined in the normal control group

The data thus obtained were analyzed according to the procedures as described in Section 1 of General Procedures.

### Results:

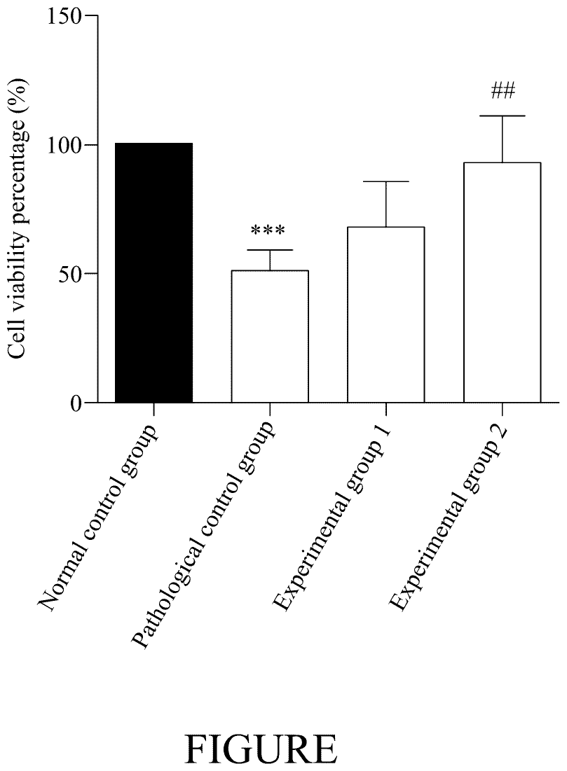

Referring to FIG. 1, compared with the normal control group, the cell viability percentage determined in the pathological control group was significantly reduced, indicating that the CSE successfully inhibited the growth of the HBEpiC. In addition, compared with the pathological control group, the cell viability percentage determined in each of the experimental groups 1 and 2 was increased significantly, in which the cell viability percentage determined in the experimental group 2 had the highest degree of increase to even reach a level approximate to the cell viability percentage determined in the normal control group. These results indicate that Lactobacillus acidophilus TW01 is capable of protecting HBEpiC against PM-induced cell death.

B. Effect of Lactobacillus acidophilus TW01 on Cell Cycle Distribution

### Experimental Procedures:

First, the CSE obtained in Section 4 of the General Experimental Materials was diluted using a suitable amount of the BEpiCM, so as to obtain a CSE solution having a CSE concentration of 10 vol %.

Subsequently, co-cultivation of the Caco-2 cells and the HBEpiC was carried out according to the procedures described in Section A of this example, except that the HBEpiC were divided into three groups, including a normal control group, a pathological control group, and an experimental group, that the HBEpiC in each of the pathological control group and the experimental group were treated with 1 mL of the aforesaid CSE solution, and that the Caco-2 cells in the experimental group were treated with 0.5 mL of the bacterial suspension 1 (having the bacterial concentration of 10⁴ CFU/mL). The Caco-2 cells in the pathological control group received no treatment. Both the HBEpiC and the Caco-2 cells in the normal control group received no treatment.

After cultivation in an incubator (37° C., 5% CO₂) for 24 hours, the Transwell inserts and the liquid in the well of each group were removed, and then the cell culture of the HBEpiC in each group was subjected to cell cycle analysis with reference to the method described in Luo S M. et al. (2021), Onco. Targets Ther., 14:1843-1855. Briefly, the cell culture of the HBEpiC was subjected to fixation with a suitable amount of 70% of ice-cold ethanol to obtain fixed cells. After that, the fixed cells were left to stand at −20° C. overnight, and then washed twice with ice-cold DPBS (containing 1% FBS), followed by staining in the dark with a DNA staining solution (prepared in a phosphate-buffered saline (PBS) containing 50 mg/mL of propidium iodide (PI) (Sigma-Aldrich Chemical Co.), 1% of Tween® 20, and 10 mg/mL of Ribonuclease A (RNase A)) at 37° C. for 45 minutes.

Thereafter, the resultant stained cells were subjected to cell cycle analysis using a Cytomics FC500 flow cytometer (Beckman Coulter Inc.), and 3×10³ stained cells were analyzed each time. Briefly, the stained cells were excited by an argon-ion laser beam at a wavelength of 488 nm to generate fluorescence, and the fluorescence intensity was then measured at a wavelength of 620 nm. The data thus obtained were analyzed using CXP Analysis software, so as to obtain a percentage of cells in each cell-cycle phase (i.e., sub-G1 phase, G1 phase, S phase, G2/M phase).

The data thus obtained were analyzed according to the procedures as described in Section 1 of General Procedures.

### Results:

Referring to Table 2, compared with the normal control group, the pathological control group had a higher proportion of cells in the sub-G1 phase, indicating that the CSE indeed promoted cell death of HBEpiC. In addition, compared with the pathological control group, the experimental group had a lower proportion of cells in the sub-G1 phase. These results indicate that Lactobacillus acidophilus TW01 is capable of protecting the HBEpiC against PM-induced cell death.

TABLE 2

Percentage of cells (%)

| | Sub-G1 | | | G2/M |
|---|---|---|---|---|
| | phase | G1 phase | S phase | phase |
| Normal control | 6.83 | 58.73 | 12.08 | 21.13 |
| group | | | | |
| Pathological control | 10.68** | 50.98 | 18.02 | 18.37 |
| group | | | | |
| Experimental group | 5.29^{##} | 57.94 | 17.58 | 17.44 |

**p < 0.01 compared with the normal control group
^{##}p < 0.01 compared with the pathological control group

Summarizing the above test results, it is clear that Lactobacillus acidophilus TW01 is capable of effectively protecting the HBEpiC against PM-induced cell death, and hence is expected to be useful for alleviating PM-induced lung injury.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, FIGURE, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating particulate matter-induced lung injury, comprising administering to a subject in need thereof a composition including an isolated strain of Lactobacillus acidophilus TW01,
wherein the isolated strain of Lactobacillus acidophilus TW01 is deposited at the Deutsche Sammlung von Mikroorganismen und Zellkulturen (DSMZ) GmbH under an accession number DSM 33990 in accordance with the Budapest Treaty.

2. The method as claimed in claim 1, wherein the composition is a food product.

3. The method as claimed in claim 1, wherein the composition is a pharmaceutical composition.

4. The method as claimed in claim 3, wherein the pharmaceutical composition is in a dosage form selected from the group consisting of an oral dosage form, a parenteral dosage form, an inhalation dosage form, and a topical dosage form.
